(19) United States
(12) Patent Application Publication
(10) Pub. No.: US 2023/0203627 A1
(43) Pub. Date: Jun. 29, 2023

(54) USE OF FERRITIC STEEL IN THE HIGH PRESSURE SECTION OF UREA PLANTS

(71) Applicant: Casale SA, Lugano (CH)

(72) Inventor: Leonardo Marrone, Mercallo (VA) (IT)

(73) Assignee: Casale SA, Lugano (CH)

(21) Appl. No.: 17/914,158

(22) PCT Filed: Mar. 16, 2021
(86) PCT No.: PCT/EP2021/056638

§ 371 Date: Sep. 23, 2022

(30) Foreign Application Priority Data
Mar. 25, 2020 (EP) 20165679.0

(51) Int. Cl. C22C 38/40; B01J 3/04; B01J 19/02; C07C 273/04

(52) U.S. Cl. CPC C22C 38/40; B01J 3/04; B01J 19/02; C07C 273/04

(57) ABSTRACT

Use of a ferritic stainless steel containing at least 23% chromium for the manufacture of components of the high-pressure urea synthesis section of a urea plant.

### FIELD OF THE INVENTION

The present invention refers to the field of materials for the manufacturing of the high-pressure equipment of urea synthesis plants.

### PRIOR ART

Urea is produced industrially by reacting ammonia and carbon dioxide at high temperature and high pressure. The reaction involves basically the formation of ammonium carbamate and its dehydration to form urea. The production of urea is known to be a challenge in terms of resistance to corrosion of the equipment because of the combination of highly corrosive substances (particularly the ammonium carbamate), high temperature and pressure.

Most of the urea production capacity currently installed use the so-called stripping process. In a stripping process, the synthesis solution leaving the reactor containing unreacted ammonia and carbon dioxide, mostly in the form of ammonium carbamate, is sent to a stripper where it is heated still at a high pressure which may be substantially the same pressure of the reactor.

During the stripping process, the ammonium carbamate decomposes into ammonia and carbon dioxide in the liquid phase and part of the liberated ammonia and carbon dioxide passes from the liquid phase to the gas phase. The stripping process therefore produces an aqueous solution of urea with a reduced content of unconverted carbamate and a gas phase containing the unconverted ammonia and carbon dioxide removed from the liquid phase. The liquid phase is normally sent to one or more stages of further recovery at a lower pressure; the gas phase is condensed at high pressure and recycled to the reactor.

The stripping process may be promoted by adding a gaseous stripping agent which may be carbon dioxide or ammonia. In absence of added stripping agent, the process is termed self-stripping.

The stripper is typically a shell-and-tube apparatus where the reaction effluent flows through the tubes, e.g. with a falling-film flow, and the tube bundle is externally heated by hot steam. The condenser is also, in most cases, a shell-and-tube apparatus. The reactor is typically a vertical pressure vessel with a suitable set of perforated plates.

The reactor, the stripper and the condenser are part of a high-pressure synthesis section also termed synthesis loop. The synthesis section may also include a scrubber for the gases vented from the reactor. These pieces of equipment operate typically at a pressure around 150 bar or more and a high temperature around 200° C. The operating conditions, in combination with the presence of the aggressive ammonium carbamate, are very demanding for the materials.

Particularly in the HP stripper, the skin temperature of the tubes can easily reach a temperature around 210° C. Therefore, the tubes of the stripper are among the most critical components because they operate under high temperature and high concentration of carbamate. The use of high-grade materials for large components like tubes and tube plates introduces a relevant cost.

For many years the ammonia-stripping and the self-stripping plants used titanium tubes for the HP stripper, as most resistant to corrosion under the urea synthesis process conditions. Nevertheless, there are many cases where tubes made of a super austenitic steel such as 25/22/2 (UNS: S31050) have been used. Being significantly cheaper than titanium, the super austenitic steel has been often considered a good alternative regardless of its lower resistance to corrosion leading to a shorter operation life.

Although titanium has proved over the time to be very resistant to chemical corrosion in urea environment, the same cannot be said for its mechanical resistance to erosion. For this reason, stainless steel UNS 31050 has usually been preferred as standard material for piping. To overcome erosion of the internal part of the titanium tubes of HP stripper, bimetallic tubes were introduced consisting of an external tube made of austenitic stainless steel and an internal tube made of zirconium. A further evolution of this concept led to full zirconium tubes or bimetallic titanium-zirconium (Ti—Zr) tubes. These materials however are very expensive.

The CO₂ stripping plants have traditionally privileged the use of special austenitic stainless steels such as UNS31050. More recently superduplex stainless steels have been used for the construction of the HP synthesis, most specifically of the HP stripper. Duplex steels are distinguished by a two-phase structure showing both ferrite and austenite. Examples of high-performance duplex steels (superduplex) are UNS S32906 and UNS S32808. Also the duplex steels are quite expensive. Compared to austenitic stainless steel, super duplex need lower content of oxygen in the liquid phase for resisting well to corrosion. On the other hand, super duplex steels are significantly more expensive than UNS31050.

In order to reduce corrosion, a known provision is to add oxygen (O₂) or a gas containing the same (e.g. air) to the loop for passivation. However materials adapted to resist corrosion without the addition, or with a lower amount, of O₂ would be preferable. Austenitic stainless steels need higher content of dissolved oxygen to be properly passivated than superduplex or even more titanium. Because of this, many urea plants feed passivation air to the HP loop. However the addition of inerts has the negative impact of worsening the performance of the synthesis loop (lower overall efficiency) and introducing a potential explosion hazard.

In general, a target corrosion rate should be not greater than around 0.1 mm/y to provide an acceptable service life of components, for example 15 or 20 years.

Due to the extremely demanding process conditions encountered in HP synthesis loop which leads to high investment costs, there is always a driving force to identify materials with a higher corrosion resistance.

As stated above, the drawback of the current materials adopted in the high pressure urea synthesis section is the cost. The bimetallic materials are not only expensive but also applicable, in practice, only to tubes, so that they do not provide a feasible solution for the manufacture of other components. The cost of duplex stainless steel is also high and is really sensitive to the cost of Nickel, which has increased significantly in recent times.

There is therefore an incentive to find alternative materials which can perform like or better than duplex steels at a lower cost and possibly without the addition of O₂ as passivation agent.

### SUMMARY OF THE INVENTION

The applicant has surprisingly found that a pure ferritic steel with a chromium content of at least 23%, preferably at least 26%, can perform under urea synthesis conditions in a manner similar to, or even better than, the above mentioned duplex steels UNS S32906 or UNS S32808 despite a significantly lower cost.

An aspect of the invention is the use of a ferritic stainless steel containing at least 23% chromium for the manufacture of components of a high-pressure urea synthesis section of a urea plant. A ferritic steel with 23% or more chromium is also termed super-ferritic. Particularly preferably, the steel contains 26% or more chromium.

The above percentages and all percentages in this description are meant as weight percentages as it is customary when referring to composition and alloy elements of steel.

### DESCRIPTION OF THE INVENTION

The invention is based on the unexpected finding that the austenite is mainly responsible for corrosion of the duplex steel in urea synthesis applications. Accordingly the applicant has found that a super-ferritic steel with 23% or more chromium and having substantially no austenitic structure can perform better than the duplex steel in a urea synthesis environment. It has been found that such super-ferritic steel may be used with low addition of oxygen O₂ for passivation or even in absence of such addition of O₂ for passivation.

The term high pressure urea synthesis section denotes the section where urea in synthesized from ammonia and carbon dioxide, including at least a urea synthesis reactor. Typically the urea synthesis section includes at least a reactor, a stripper and a condenser. According to the kind of urea plant, it may also include a scrubber.

The components of a high pressure urea synthesis section are known to a person skilled in the field of urea. Particularly the term components of a high pressure urea section may include any of: urea synthesis reactor, high-pressure stripper, high-pressure condenser, high-pressure scrubber, related connection piping and internals. For example the internals may include tubes and/or tube sheets of a shell-and-tube stripper or of a shell-and-tube condenser. The internals may also include internal plates of a reactor and other internal piping, baffles and similar. The steel of the invention may also be used for manufacturing the pressure vessel of any of the above mentioned equipment.

The high pressure of urea synthesis pressure is generally above 100 bar and typically in the range 100 to 200 bar, more preferably in the range 140 to 180 bar.

Another great advantage of the invention is the reduced cost compared to the duplex steels and bimetallic materials. The superferritic steel can be employed for all crucial components in the urea synthesis section including vessel, tubes, tube plates etc.

Preferred aspects are recited in the dependent claims.

According to an embodiment, the steel of the present invention contains no more than 3.5% by weight of nickel. Preferably the steel contains some nickel, although not more than said 3.5% by weight. For example the steel may contain 0.1% to 3.5% by weight of nickel.

A preferred embodiment includes using the steel of the present invention in absence of an addition of oxygen (O₂) or of an oxygen-containing gas for passivation, for example passivation air introduced into the synthesis loop.

Accordingly an aspect of the invention is a process for the synthesis of urea wherein urea is synthesized in a high-pressure synthesis section and wherein one or more components of said section are made of a ferritic stainless steel as above mentioned, and wherein no addition of oxygen or oxygen-containing gas is provided for the passivation of said components made of the ferritic stainless steel.

Still further preferred conditions of use of the steel of the invention include: the operating temperature is greater than the transition temperature. Said transition temperature may be 100° C. or about 100° C. in exemplary applications. A particularly preferred ferritic steel for the use of the present invention is according to UNS S44600. Another particularly preferred ferritic steel is according to UNS S44660.

A steel according to the designation UNS S44600 may contain (% by weight):

| Iron, Fe | around 73 |
|---|---|
| Chromium, Cr | 23.0-27.0 |
| Nitrogen, N | 0.17 |
| Manganese, Mn | 1.50 |
| Silicon, Si | ≤1.0 |
| Nickel, Ni | 0.25 |
| Carbon, C | ≤0.20 |
| Phosphorous, P | ≤0.04 |
| Sulfur, S | ≤0.03 |

A steel according to the designation UNS S44660 may contain (% by weight):

| | |
|---|---|
| Iron, Fe | 60.4-71-0 |
| Chromium, Cr | 25.0-28.0 |
| Nitrogen, N | ≤0.04 |
| Molybdenum Mo | 3.0-4.0 |
| Nickel | 1.0-3.5 |
| Manganese, Mn | ≤1.0 |
| Silicon, Si | ≤1.0 |
| Carbon, C | ≤0.03 |
| Phosphorous, P | ≤0.04 |
| Sulfur, S | ≤0.03 |
| Ti, Nb | 0.1 to 1.0 |

The super-ferritic steel may be used for the manufacture of pressure vessel internals of any of: a reactor, a stripper, a condenser, a scrubber in the high-pressure synthesis section. Particularly it may be used for the manufacture of a tube sheet and/or of a tube plate of a shell-and-tube stripper or of a shell-and-tube condenser in the high-pressure synthesis section.

The ferritic chromium steels are less tough than austenitic stainless steels at low temperature. The term transition temperature denotes the ductile to brittle transition temperature, i.e. the temperature below which the toughness of the material drops down and the material becomes brittle. In the ferritic steels used in the invention, said transition can occur at 100° C. or about 100° C. The preferred applications have an operating temperature of the material higher than its transition temperature.

An aspect of the invention is also an equipment for a high pressure urea synthesis section wherein the equipment includes at least one component made with a ferritic steel as described above. Particularly the equipment may be any of: a reactor, a stripper, a condenser, a scrubber of the high-pressure synthesis section. The equipment may have no addition of O₂ for passivation.

Test Data

The super-ferritic steels S44600 and S44660 were tested in an autoclave where the conditions typical of a high pressure urea synthesis section were simulated in absence of oxygen. The test conditions were as follows:

| N/C (ammonia/CO2) | ratio: 3.2 | |
|---|---|---|
| H/C (water/CO2) | ratio: 0.8 | |
| Temperature: | 211° | C. |
| Exposure time: | 12 | days |
| Pressure | 240 | bar. |

As reference material, a superduplex steel S32906 was tested under the same conditions. The following rates of corrosion (mm/y) were detected:

| | |
|---|---|
| S44660 | 0.04 |
| S44600 | 0.05 |
| S32906 | 0.20. |

The test demonstrated that the superferritic steels are superior to the reference superduplex under typical conditions found in the equipment of a urea synthesis section.

1) Use of a ferritic stainless steel containing at least 23% by weight chromium for the manufacture of components of a high-pressure urea synthesis section of a urea plant.

2) The use of claim 1 wherein the steel contains at least 26% by weight chromium.

3) The use of claim 2 wherein the steel contains 26% to 30% by weight chromium.

4) The use of any of the previous claims wherein the steel contains no more than 3.5% by weight of nickel.

5) The use of claim 1 wherein the steel is UNS S44600.

6) The use of claim 1 wherein the steel is UNS S44660.

7) The use according to any of the previous claims, wherein the steel is used for the manufacture of a tube sheet and/or of a tube plate of a shell-and-tube stripper or of a shell-and-tube condenser in the high-pressure synthesis section.

8) The use of any of the previous claims, wherein the steel is used for the manufacture of pressure vessel internals of any of: a reactor, a stripper, a condenser, a scrubber in the high-pressure synthesis section.

9) The use of any of the previous claims wherein the steel operates in absence of an addition of oxygen (O₂) or of an oxygen-containing gas for passivation.

10) The use of any of the previous claims wherein the operating temperature of the material is greater than its transition temperature.

11) The use of claim 10, wherein the transition temperature is 100° C. or about 100° C.

12) An equipment for a high pressure urea synthesis section wherein the equipment includes at least one component made with a ferritic steel as described in any of claims 1 to 6.

13) An equipment according to claim 12, wherein the equipment is any of: a reactor, a stripper, a condenser, a scrubber of the high-pressure synthesis section.

14) An equipment according to claim 12 or 13 having no means arranged for addition of O₂ or of a gas containing O₂ for passivation.
